(19) United States
(12) Patent Application Publication
(10) Pub. No.: US 2021/0395807 A1
(43) Pub. Date: Dec. 23, 2021

(54) SYSTEM AND METHOD FOR DATA ANALYSIS IN QUANTITATIVE PCR MEASUREMENTS

(71) Applicant: Government of the United States of America, as represented by the Secretary of Commerce, Gaithersburg, MD (US)

(72) Inventors: Paul Nathan Patrone, Washington Grove, MD (US); Anthony Jose Kearsley, Hanover, MD (US); Erica Lee Romsos, Ijamsville, MD (US); Peter Michael Vallone, Potomac, MD (US)

(21) Appl. No.: 17/350,666

(22) Filed: Jun. 17, 2021

Related U.S. Application Data
(60) Provisional application No. 63/040,310, filed on Jun. 17, 2020.

(51) Int. Cl. C12Q 1/6844; C12Q 1/6806

(52) U.S. Cl. CPC C12Q 1/6844; C12Q 1/6806

(57) ABSTRACT

Embodiments of the present invention relate to a system and method for determining quantity of target nucleic acid sequence in a sample. During a PCR-based amplification reaction, fluorescence intensity signals are acquired that form an amplification profile from which an exponential amplification region is desirably identified. In determining the exponential region, embodiments of the present invention determine a fluorescence threshold by background subtraction, test the feasibility of matching a signal to a reference curve and, in the event the feasibility test is successful, determine the matching parameters that quantify the initial amplicon number, and signal detection that reduces systematic errors in the measurements and increase the sensitivity of the measurement by decreasing the apparent noise-floor.

### CROSS-REFERENCE TO RELATED APPLICATION

This application claims the benefit of priority from U.S. Provisional Patent Application Ser. No. 63/043,310, filed on Jun. 17, 2020, the disclosure of which is incorporated herein by reference.

### STATEMENT REGARDING FEDERAL RIGHTS

The invention described herein was made with United States Government support from the National Institute of Standards and Technology (NIST), an agency of the United States Department of Commerce. The United States Government has certain rights in the invention.

### FIELD OF THE INVENTION

The present invention relates generally to nucleic acid analysis, and more particularly, to a system and method for evaluating results obtained from quantitative amplification reactions.

### BACKGROUND OF THE INVENTION

Quantitative polymerase chain-reaction (qPCR) measurements are a mainstay diagnostic tool for early disease detection. qPCR technique involves iterating or “cycling” a PCR reaction to double the amount of a target DNA segment in a sample and detecting fluorescence emission signals corresponding to new copy of target DNA generated during each cycle of the PCR reaction. For example, qPCR can be used to detect viral particles in a patient sample by targeting a specific genetic sequence associated with the viral genome and exponentially amplifying the corresponding DNA, which is observed indirectly via the increasing amplitude of fluorescence. Fluorescent emission signals are generally not detected until the concentrations of the duplicated target DNA reach certain levels and standard amplification protocols of about 40 cycles provide sufficient concentrations to detect a single target DNA strand. Further, fluorescence emission signal measurements must account for background sources of light that obscure signals of interest.

Typical approaches for the analysis of data obtained from qPCR measurements rely on local empirical fits of the amplification curves at low cycle numbers that are generally before the onset of exponential growth. This assumes that the local behavior can be extrapolated to high cycle numbers to model the global background structure. However, current techniques to subtract background signals from qPCR measurement signals include empirical polynomial models that are often extrapolated to correct data outside the fit region. Such techniques have been found to introduce systematic errors into measurements and may decrease the sensitivity of diagnostic protocols by increasing the apparent noise-floor. This problem is further compounded by data analysis techniques that rely on subjective thresholds that an amplification curve (as a function of cycle number) must surpass to be considered a true positive signal and assumptions that baseline corrections so extracted will be valid for high cycle numbers. Because these thresholds must be significantly larger than the apparent noise-floor, systematic errors unnecessarily force thresholds higher and increase the probability of false-negatives. Moreover, threshold-based analysis methods do not directly test that the signal manifests exponential growth and, in extreme cases, systematic background errors may lead to false positives. Even if the background signals are correctly subtracted, thresholds may still be unable to detect noisy but statistically significant signals.

Accordingly, there is a need for an improved method for analyzing data generated by qPCR measurements to detect low initial concentrations of target nucleic acid while improving the quantitative accuracy and reproducibility of the analysis. There is also a need for a baseline subtraction technique in qPCR measurements that avoids the use of empirical models by directly leveraging the behavior of appropriate control experiments. Moreover, there is also need for methods that can identify mutated strains of variants without the need for full genetic sequencing.

### SUMMARY OF THE INVENTION

Embodiments of the present invention relate to a system and method for determining quantity of target nucleic acid sequence in a sample. Embodiments of the present invention also relate to methods for identifying and determining initial quantity of target amplicon in a sample. During a PCR-based amplification reaction, fluorescence intensity signals are acquired that form an amplification profile from which an exponential amplification region is desirably identified. In determining the exponential region, embodiments of the present invention determine a fluorescence threshold by background subtraction, test the feasibility of matching a signal to a reference curve and, in the event the feasibility test is successful, determine the matching parameters that quantify the initial amplicon number, and signal detection that reduces systematic errors in the measurements and increase the sensitivity of the measurement by decreasing the apparent noise-floor.

Accordingly, embodiments of the present invention relate to a method for determining a quantity of a target nucleic acid in a sample, the method comprising performing a first amplification reaction on a plurality of control samples; receiving a plurality of first optical signals as a function of a first cycle number for the plurality of the control samples, wherein the plurality of the first optical signals comprises a plurality of first background signals; performing a second amplification reaction on the sample; receiving a plurality of second optical signals as a function of a second cycle number for the target nucleic acid in the sample, wherein the plurality of the second optical signals comprise at least one of the plurality of the first background signals and a plurality of second background signals; optimizing, using a processor, the plurality of the first background signals from the plurality of the first optical signals for each of the plurality of the control samples, wherein the optimizing the plurality of the first background signals comprises subtracting the plurality of the first background signals from the plurality of the second optical signals received to obtain a mean baseline, wherein the mean baseline corresponds to the second cycle number such that the plurality of the second optical signals corresponds to a predetermined value; validating, using the processor, each of the plurality of the second optical signals against at least one of a plurality of reference amplification curves; and computing, using the processor, the quantity of the target nucleic acid in the sample from the validated plurality of the second optical signals obtained as the function of the second cycle number for the target nucleic acid in the sample, wherein computing the quantity of the target nucleic acid in the sample from the validated plurality of the second optical signals comprises performing an affine transformation to apply at least one of a linear transformation and a translation on the validated plurality of the second optical signals. More particularly, the control sample is an extraction blank or a non-template control. In one embodiment of the present invention, the sample is a pre-amplified DNA sample. In another embodiment of the present invention, the sample is a pre-amplified RNA sample.

In one embodiment of the present invention, the receiving the plurality of the first optical signals as the function of the first cycle number comprises detecting the plurality of the first optical signals for each of the plurality of the control samples at each cycle of the first amplification reaction, wherein each cycle of the first amplification reaction corresponds to the first cycle number; and plotting the plurality of the first optical signals detected as the function of the first cycle number for each of the plurality of the control samples to obtain a first amplification curve, wherein the first amplification curve represents a ratio of each of the plurality of the first optical signals to a passive reporter dye optical signal as the function of the first cycle number.

In another embodiment of the present invention, the receiving the plurality of the second optical signals as the function of the second cycle number comprises detecting the plurality of the second optical signals for the target nucleic acid in the sample at each cycle of the second amplification reaction, wherein each cycle of the second amplification reaction corresponds to the second cycle number; and plotting the plurality of the second optical signals detected as the function of the second cycle number for the target nucleic acid in the sample to obtain a second amplification curve, wherein the second amplification curve represents a ratio of each of the plurality of the second optical signals to a passive reporter dye optical signal as the function of the second cycle number.

In one embodiment of the present invention, the validating each of the plurality of the second optical signals against the at least one of the plurality of the reference amplification curves comprises: generating at least the one of the plurality of the reference amplification curves, wherein the at least one of the plurality of the reference amplification curves comprises a plurality of third optical signals corresponding to a reference sample, wherein the at least one of the plurality of the reference amplification curves comprises the plurality of the third optical signals corresponding to a background region, an exponential growth region, and a plateau region; projecting each of the plurality of the second optical signals on to the at least one of the plurality of the reference amplification signals, wherein the projecting each of the plurality of the second optical signals on to the at least one of the plurality of the reference amplification curves comprises determining whether each of the plurality of the second optical signals obtained as the function of the second cycle number for the target nucleic acid in the sample collapses on to the at least one of the plurality of the reference amplification curves; and determining a threshold value for the plurality of the second optical signals projected on to the at least one of the plurality of the reference amplification curves.

In some embodiments, the validating each of the plurality of the second optical signals against the at least one of the plurality of the reference amplification curves further comprises plotting the plurality of the second optical signals projected on to the at least one of the plurality of the reference amplification curves to obtain a validated reference amplification curve.

Some embodiments of the present invention further include detecting the plurality of third optical signals for a plurality of amplicons, wherein each of the plurality of the third optical signals correspond to an optical signal from at least one fluorescent dye.

In one embodiment of the present invention, the linear transformation includes scaling, and the translation is selected from a group comprising a horizontal shift and a vertical scaling.

In another embodiment of the present invention, the validating each of the plurality of second optical signals against at least one of a plurality of reference amplification curves includes generating the plurality of the reference amplification curves, wherein each of the plurality of the reference amplification curves comprises a plurality of third optical signals corresponding to a reference sample, wherein each of the plurality of the reference amplification curves comprises the plurality of the third optical signals corresponding to a background region, an exponential growth region, and a plateau region; projecting the plurality of the second optical signals onto the plurality of the reference amplification curves, wherein projecting the plurality of the second optical signals onto the plurality of the reference amplification curves comprises determining whether each of the plurality of the second optical signal obtained as the function of the second cycle number for the target nucleic acid in the sample collapses on to at least one of the plurality of reference amplification curves; and determining a threshold value for the plurality of the second optical signals projected on to the at least one of the plurality of the reference amplification curves.

Some embodiments of the present invention further include normalizing the plurality of the first optical signals and the plurality of the second optical signals, wherein normalizing the plurality of the first and the second optical signals comprises determining a ratio of each of the plurality of the first and the second optical signals to a passive reporter dye optical signal.

Another embodiment of the present invention relates to a method for determining a quantity of a target nucleic acid in a sample, including performing a first amplification reaction on a plurality of control samples; receiving a first amplification curve representing a plurality of first optical signals as a function of a first cycle number for each of the plurality of control samples at each cycle of the first amplification reaction, wherein the plurality of the first optical signals comprises a plurality of background signals; performing a second amplification reaction on the sample; receiving a second amplification curve representing a plurality of second optical signals as a function of a second cycle number for the target nucleic acid in the sample at each cycle of the second amplification reaction; optimizing, using the processor, the plurality of the background signals from the received plurality of first optical signals for each of the plurality of the control samples, wherein the optimizing the plurality of the background signals comprises subtracting the plurality of the background signals from the plurality of the second optical signals received to obtain a mean baseline, wherein the mean baseline corresponds to the second cycle number such that the plurality of the second optical signals corresponds to a predetermined value; validating, using the processor, each of the plurality of second optical signals detected against a plurality of reference amplification curves, wherein validating each of the plurality of the second optical signals includes: receiving the plurality of the reference amplification curves, wherein each of the plurality of the reference amplification curves comprises a plurality of third optical signals corresponding to a reference sample, wherein each of the plurality of the reference amplification curves comprises the plurality of the third optical signals corresponding to a background region, an exponential growth region, and a plateau region; projecting each of the plurality of the second optical signals on to the plurality of the reference amplification curves; and determining a threshold for the plurality of the second optical signals projected onto at least one of the plurality of the reference amplification curves; and performing, using the processor, an affine transformation on the validated plurality of the second optical signals to determine the quantity of the target nucleic acid in the sample, wherein the affine transformation comprises at least one of a linear transformation and a translation. More particularly, the control sample is an extraction blank or a non-template control. In one embodiment of the present invention, the sample is a pre-amplified DNA sample. In another embodiment of the present invention, the sample is a pre-amplified RNA sample.

In some embodiments of the present invention, the projecting each of the plurality of the second optical signals on to the plurality of the reference amplification curves includes determining whether each of the plurality of the second optical signal obtained as the function of the second cycle number for the target nucleic acid in the sample collapses on to at the least one of the plurality of the reference amplification curves.

Embodiments of the present invention also relate to a system of determining quantity of a target nucleic acid in a sample, including a reaction chamber for performing a first amplification reaction on a plurality of control samples and a second amplification reaction on the sample; a detector for detecting a plurality of first optical signals as a function of a first cycle number for the plurality of the control samples at each cycle of the first amplification reaction and a plurality of second optical signals as a function of a second cycle number for the target nucleic acid in the sample at each cycle of the second amplification reaction, wherein the plurality of the first optical signals comprises a plurality of background signals; and a processor comprising a memory for implementing program instructions of a computer-readable medium, wherein said program instructions include: optimizing the plurality of the background signals from the received plurality of the first optical signals for each of the plurality of the control samples, wherein the optimizing the plurality of the background signals comprises subtracting the plurality of the background signals from each of the plurality of the second optical signals received to obtain a mean baseline, wherein the mean baseline corresponds to the second cycle number such that the plurality of the second optical signals correspond to a predetermined value; validating each of the plurality of the second optical signals detected against a plurality of reference amplification curves, wherein the validating each of the plurality of the second optical signals comprises projecting each of the plurality of the second optical signals on to the plurality of the reference amplification curves to determine a threshold for the plurality of the second optical signals; and computing an initial quantity of the target nucleic acid in the sample from the validated plurality of the second optical signals obtained as the function of the second cycle number for the target nucleic acid in the sample, wherein computing the initial quantity of the target nucleic acid in the sample from the validated plurality of the second optical signals comprises performing an affine transformation on the validated plurality of the second optical signals, wherein the affine transformation comprises at least one of a linear transformation and a translation.

### BRIEF DESCRIPTION OF DRAWINGS

FIG. 1 is a flowchart illustrating an overview of a data analysis method in accordance with an embodiment of the present invention.

FIG. 2 illustrates an exemplary plot showing fluorescence emission signal intensities as a function of cycle number for a PCR reaction.

FIG. 3 illustrates an exemplary plot showing fluorescence emission signal intensities as a function of cycle number for extraction blanks or non-template controls.

FIG. 4 illustrates an embodiment of a method for validating fluorescence emission signal intensities against a reference amplification signal wherein constrained optimization compares a test signal to a reference or “master” amplification curve.

FIG. 5 illustrates an exemplary analysis applied to RT-qPCR measurements of N1 fragment of a SARS-CoV-2 RNA construct using methods in accordance with embodiments of the present invention.

FIG. 6 illustrates exemplary data collapse using optimization methods in accordance with an embodiment of the present invention.

FIG. 7 illustrates differences between reference and transformed curves for the collection of datasets shown in FIG. 6.

FIG. 8 illustrates a plot including truncated data used to test for feasibility of data collapse using a lower threshold.

FIG. 9 illustrates data collapse of the amplification curves shown in FIG. 8.

FIG. 10 illustrates exemplary transformations of non-template control data for τ=0 (low-threshold) and τ=μ+5σ (high-threshold), where σ was computed individually for each non-template control.

FIG. 11 illustrates feasibility of transforming amplification curves and non-template controls as a function of the mean threshold τ.

FIG. 12 illustrates the results of affine analysis performed on exemplary datasets spanning 3.5 years.

FIG. 13 illustrates an exemplary analysis applied to RT-qPCR measurements of N2 fragment of a SARS-CoV-2 RNA construct using methods in accordance with embodiments of the present invention and a comparison of measurements of N2 fragment of a SARS-CoV-2 RNA construct with measurements of N1 fragment of a SARS-CoV-2 RNA construct.

FIG. 14 illustrates an exemplary system for performing quantitative PCR in conjunction with the quantitation method in accordance with embodiments of the present invention.

### DETAILED DESCRIPTION

While the making and using of various embodiments of the present invention are discussed in detail below, it should be appreciated that the present invention provides many applicable inventive concepts which can be embodied in a wide variety of specific contexts. The specific embodiments discussed herein are merely illustrative of specific ways to make and use the invention, and do not delimit the scope of the present invention. Reference will now be made to the drawings wherein like numerals refer to like elements throughout.

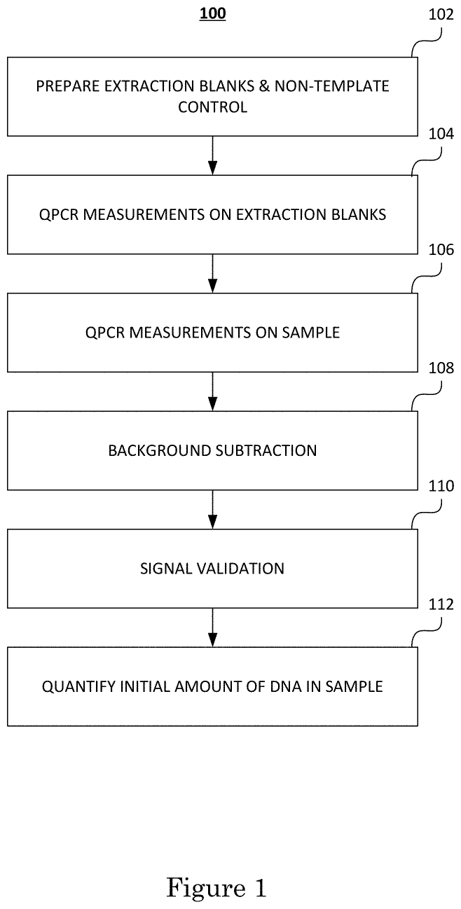

Referring now to the drawings, and more particularly, to FIG. 1, there is shown a method for determining quantity of target nucleic acid in a biological sample, generally designated 100, which comprises embodiments of the present invention.

As used herein, a nucleic acid “sequence” means a nucleic acid base sequence of a polynucleotide. Unless otherwise indicated or apparent from context, bases or sequence elements are presented in the order 5′ to 3′ as they appear in a polynucleotide.

A “polynucleotide” or “nucleic acid” includes any form of RNA or DNA, including, for example, genomic DNA; complementary DNA (cDNA), which is a DNA representation of messenger RNA (mRNA), usually obtained by reverse transcription of mRNA; and DNA molecules produced synthetically or by amplification. Polynucleotides include nucleic acids comprising non-standard bases (e.g., inosine). A polynucleotide in accordance with the invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages; positive backbones; non-ionic backbones, and non-ribose backbones. Polynucleotides may be single-stranded or double-stranded.

As used herein, “amplification” of a nucleic acid sequence has its usual meaning, and refers to in vitro techniques for enzymatically increasing the number of copies of a target sequence. Amplification methods include both asymmetric methods (in which the predominant product is single-stranded) and conventional methods (in which the predominant product is double-stranded).

The terms “amplicon” and “amplification product” are used interchangeably and have their usual meaning in the art. The grammatically singular term, “amplicon,” can refer to many identical copies of an amplification product. Moreover, reference to an “amplicon” encompasses both a molecule produced in an amplification step and identical molecules produced in subsequent amplification steps (such as, but not limited to, amplification products produced in subsequent rounds of a PCR amplification). Moreover, the term “amplification” may refer to cycles of denaturation, annealing and extension, and does not require geometric or exponential increase of a sequence.

As used herein, “quantitative PCR” or “qPCR” refers to quantitative real-time polymerase chain reaction (PCR), which is also known as “real-time PCR” or “kinetic polymerase chain reaction.”

A “reagent” refers broadly to any agent used in a reaction, other than the analyte (e.g., protein being analyzed). Illustrative reagents for a nucleic acid amplification or extension reaction include, but are not limited to, buffer, metal ions, polymerase primers, template nucleic acid, nucleotides, labels, dyes, nucleases, and the like. Reagents for enzyme reactions include, for example, substrates, cofactors, buffer, metal ions, inhibitors, and activators.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

As used herein, a “sample” refers to any substance comprising a target nucleic acid of interest (e.g., a target polynucleotide or a target polypeptide). The term “sample” thus can include a sample of polynucleotide (genomic DNA, cDNA, RNA) and/or polypeptide such as can be found in a cell, tissue, bodily fluid, tumor, organ, organism, samples of in vitro cell culture constituents, an environmental sample, or industrial sample (e.g., a commercial food product, an industrial waste product, and the like). Exemplary bodily fluid includes plasma, serum, spinal fluid, lymph fluid, synovial fluid, urine, tears, stool, external secretions of the skin, respiratory, intestinal and genitourinary tracts, saliva, blood, and the like.

As used herein, “threshold” or “threshold value” refers to a fluorescence signal value required for measurement by a fluorescence detector.

As used herein, “quantification cycle” or “Cq” refers to the PCR cycle number at which a sample's amplification curve intersects a threshold.

As used herein, “extraction blank” or “EB” refers to a negative control in which water (or a clean swab) is used instead of a sample contain cells or DNA and all steps for nucleic acid extraction are performed as if it were a normal sample.

As used herein, “non-template control” or “NTC” refers to all reagents used in the PCR except the template nucleic acid but including the internal control.

According to various embodiments, quantitation method 100 commences by collecting samples and preparing a plurality of extraction blanks at step 102. In an alternate embodiment of the present invention, quantitation method 100 commences by preparing a plurality of non-template controls at step 102. At step 104, qPCR measurements are performed to obtain fluorescence emission signal intensities as a function of cycle number for each of the plurality of extraction blanks or non-template controls from step 102. At step 106, qPCR measurements are performed on a plurality of samples containing a target nucleic acid to obtain fluorescence emission signal intensities as a function of cycle number for the target nucleic acid in each of the samples.

Conventional qPCR techniques may be used to perform qPCR measurements at step 106. During a typical qPCR process, amplification of a target template DNA strand proceeds through a series of temperature regulated cycles using the activity of a thermostable enzyme and a sequence specific primer set. At an appropriate temperature, the primers hybridize to portions of the target DNA strand and the enzyme successively adds a plurality of nucleotide bases to elongate the primer which results in the production of progeny (daughter) strands. Each progeny strand possesses a complementary composition relative to the target template strand from which it was derived and can serve as a template in subsequent reaction cycles.

When applying quantitative methods to PCR-based technologies a fluorescent probe or other detectable reporter construct is incorporated into the reaction to provide a means for determining the progress of the target template DNA amplification. In the case of a fluorescent probe, the reaction fluoresces in relative proportion to the quantity of DNA product produced. The reaction kinetics typically change during amplification of the target such that the amount of product formed does not necessarily increase at a constant rate. The quantity or intensity of fluorescence may then be correlated with the amount of product formed in the reaction. In some embodiments of the present invention, a single fluorescent probe or other detectable reporter construct is used for detecting a plurality of amplicons.

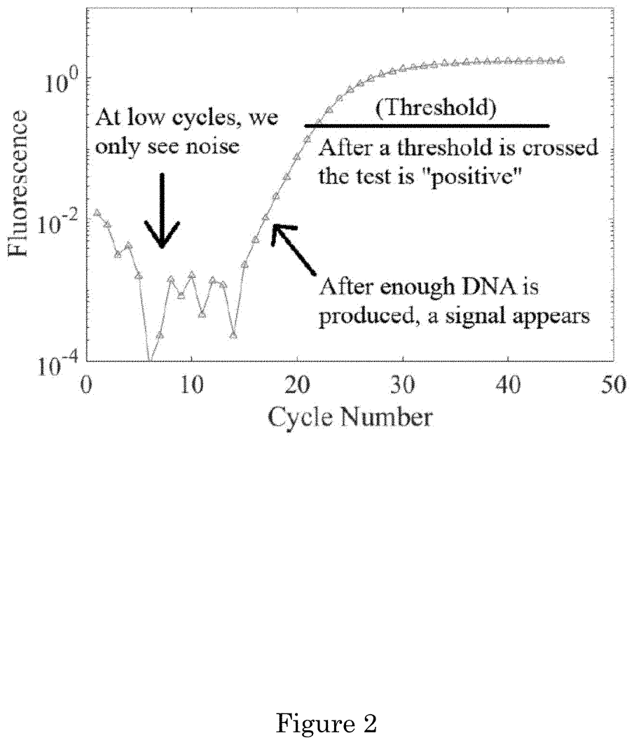

FIG. 2 illustrates an exemplary amplification curve for a PCR reaction, where fluorescence intensity values are plotted against cycle number of the PCR reaction for which the reaction fluorescence is observed. The amplification curve typically includes a noise region followed by an exponential region and then by a plateau region, as shown in FIG. 2. The noise region typically corresponds to the earlier cycles of the reaction, where the observed fluorescence may be erratic and the amount of fluorescence produced by the amplification reaction cannot be readily distinguished from background and/or non-specific fluorescence produced by the instrumentation and detection equipment. It is typically desirable to distinguish the noise region from other regions of the amplification curve, which may more accurately reflect the true fluorescence of the desired products of the reaction. It is also typically desired to normalize the data and determine a baseline that is fit to the data extending through the noise region. The baseline may be subtracted from raw fluorescence data to convert the data into corrected measurements of fluorescence intensity. The growth portion reflects the onset of amplification, which occurs at the end of the baseline obtained using the noise region. A growth portion may have exponential, sigmoidal, high order polynomial, or other type of logistic function or logistic curve that models growth.

It is desirable to identify a transition point at the end of the baseline, which is referred to commonly as the elbow value (where the fluorescence signal begins to increase exponentially) for understanding characteristics of the PCR amplification process. After the application of a baseline, a quantification cycle value (Cq) may be used as a measure of efficiency of the PCR process. For example, typically a defined signal threshold is determined for all reactions to be analyzed and the number of cycles (Cq) required to reach this threshold value is determined for the target nucleic acid as well as for reference nucleic acids such as a standard or housekeeping gene. The absolute or relative copy numbers of the target molecule can be determined on the basis of the Cq values obtained for the target nucleic acid and the reference nucleic acid.

As shown in FIG. 2, the exponential region may be followed by a plateau region where the reaction is no longer increasing in an exponential manner. Typically, the plateau region occurs in the later cycles of the reaction and results from depletion of primers or reagents. When performing quantitation calculations, it is useful to distinguish the exponential region from the plateau region to avoid erroneous or non-representative quantitation values. Methods in accordance with the present invention as described herein distinguish the noise region from the exponential region and the plateau region.

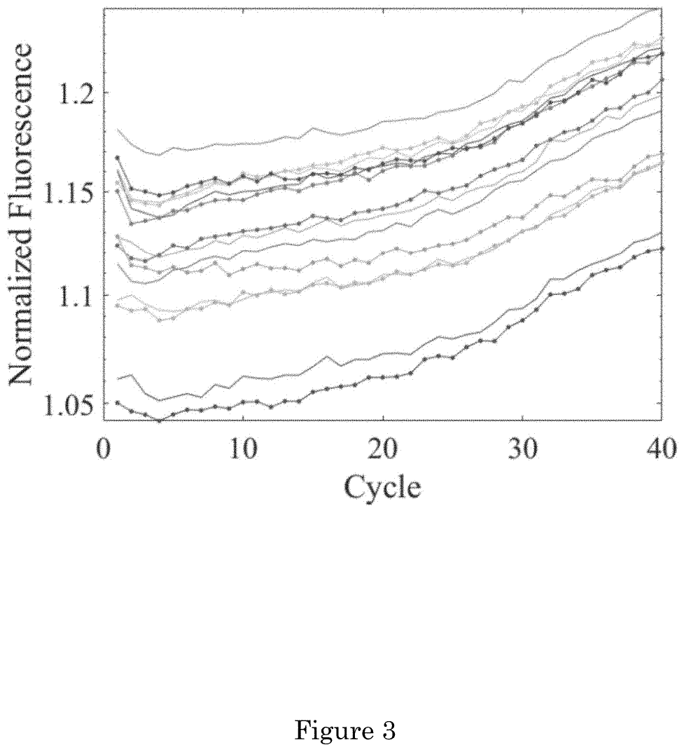

FIG. 3 illustrates an exemplary plot showing fluorescence emission signal intensities as a function of cycle number for extraction blanks or non-template controls. For the cycles typically considered to be within the noise region (cycles 3 to 15), the curves exhibit an approximately linear behavior on a logarithmic scale, which corresponds to exponential growth on a linear scale and changes in the regions typically associated with exponential growth and plateau (cycles 25 to 40). When performing background subtraction, it is useful to account for this change in behavior of the extraction blanks or non-template controls to avoid over- or under-correcting the amplification curves in the exponential and plateau regions.

As an initial preconditioning step, all fluorescence emission signals, including signals obtained from extraction blanks or non-template controls, obtained are normalized at step 108 so that the maximum fluorescence emission signal intensity of any amplification curve is of order 1. Normalization of fluorescence emission signals is generally dependent on the amplification chemistry.

In one embodiment of the present invention, normalization of fluorescence emission signals can be accomplished by dividing the raw fluorescence emission signal intensities by fluorescence emission signal intensities of a passive reporter dye to obtain a ratio denoting normalized reporter. A normalized reporter value can be calculated for every cycle and is typically plotted as an available view of the qPCR amplification data. Exemplary passive reporter dyes that can be used for normalizing fluorescence emission signals include 6-carboxyl-X-Rhodamine (ROX), carboxytetramethylrhodamine (TAMRA™), a Black Hole Quencher (BHQ) dye, an OREGON GREEN® dye (Molecular Probes), 4-dimethylaminoazobenzene-4′-sulfonyl chloride (DAB SYL); tetrachlorofluorescein (TET), and the like. In another embodiment of the present invention, normalization of fluorescence emission signals can be accomplished without a passive reference dye by dividing all signals by an arbitrary constant such that the maximum signals are on the numerical scale of unity.

Measurement signals obtained from extraction blanks at step 104 may be used as background signals for subsequent measurements of samples. As further shown in FIG. 3, fluorescence emission signal intensities as a function of cycle number for extraction blanks or non-template controls is non-linear and varies for each of the extraction blank or non-template control measured. Accordingly, background signals obtained at step 104 are not subtracted from signals obtained for samples. Instead, background signals obtained from measurements of extraction blanks (or non-template controls) at step 104 are optimized at step 108 to determine the amount of background that, when removed from sample signals obtained at step 106, minimizes the mean baseline and its variation. This baseline corresponds to the first few cycles of the amplification curve where there are too few fluorophores to detect.

In one embodiment of the present invention, optimization of background signals at step 108 can be performed using an approach that leverages information obtained from extraction blanks (or non-template controls). This approach postulates that the fluorescence signal can be expressed in the form

dₙ=sₙ+βbₙc+ηₙ (1)

where sₙ is the “true,” noiseless signal, bₙ is the average over extraction blank signals (or non-template controls), the β, c are unknown parameters quantifying the amount of systematic background effects and offset (e.g. due to photodetector dark currents) contributing to the measured signal, and η is zero-mean, delta-correlated background noise; that is, the average over realizations of η satisfies (ηₙηₙ′)=σ²δ_{n,n′}, where δ_{n,n′} is the Kronecker delta and σ² is independent of n.

Next, an objective function of the form shown below is minimized with respect to 13 and c, which determines optimal values of these parameters,

$${ℒ}_{b} ( β , c ) = {ϵ ( β - 1 )}^{2} + {\sum }_{n = {N}_{0}}^{{N}_{h}} \frac{{( {d}_{n} - β {b}_{n} - c )}^{2}}{Δ N - 1} + {[ \frac{1}{Δ N} {\sum }_{n = {N}_{0}}^{{N}_{h}} ( {d}_{n} - β {b}_{n} - c ) ]}^{2} \qquad ( 2 )$$

wherein, ϵ is a regularization parameter satisfying 0<ϵ<<1, ΔN=Nₕ−N0, and N₀ and Nₕ are lower and upper cycles for which sₙ is expected to be zero. In one embodiment of the present invention, c is set to 10⁻³. N₀ is set to 5 to accommodate transient effects associated with the first few cycles. Nₕ is determined iteratively by: (i) setting Nₕ=15 and minimizing Eq. (2); (ii) estimating C_{q} as the (integer) cycle closest to a threshold of 0.1; and (iii) defining Nₕ as the nearest integer to C_{q}−z. In an exemplary embodiment of the present invention, z is set to 6, which, assuming perfect amplification, corresponds to Nₕ falling within the cycles for which dₙ is dominated by the noise ηₙ. z and the corresponding threshold can be changed as needed, provided z is large enough to ensure that the above criterion is satisfied. Optimization of Eq. (2) amounts to calculating the amount of extraction blank signal that, when subtracted from dₙ, minimizes the mean-squared and variance of sₙ in the region where it is expected to be dominated by the noise η.

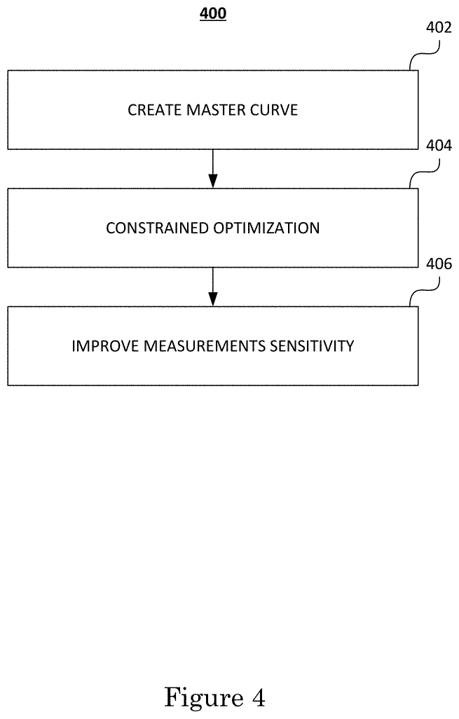

Referring to FIG. 1, at step 110, fluorescence emission signal intensities obtained at step 106, as a function of cycle number for the target nucleic acid in each of the samples, are validated against a reference amplification signal. FIG. 4 illustrates an embodiment of a method 400 for validating the fluorescence emission signal intensities obtained from samples at step 106 against a reference amplification signal, wherein constrained optimization compares a test signal to a reference or “master” amplification curve. An appropriate reference sample should be considered for accurate quantification of the nucleic acid expression because the quantification cycle (C_{q}) of the target nucleic acid is compared to the C_{q} of the reference sample.

In one embodiment of the present invention, validation method 400 creates a reference or “master” amplification curve at step 402 that exhibits exponential growth at an early cycle number. In another embodiment of the present invention, validation method 400 creates a library of reference or master amplification curves at step 402. In some embodiments of the present invention, the library of reference or master amplification curves may be from a predetermined collection of reference samples. In some embodiments of the present invention, the reference amplification curve may include data from all phases of the PCR reaction, including background noise or no detectable signal, exponential growth, and plateau. In other embodiments of the present invention, the reference amplification curve is typically a raw measurement signal from a non-template control followed by data smoothing in regions of the data where the exponential phase meets the noise floor.

In an embodiment in accordance with the present invention, a reference signal δ is obtained by fitting a cubic spline to the amplification curve obtained from a sample with the smallest Cq value determined by setting the threshold at 0.1. Moreover, comparison of amplification curves with initial template numbers that do not differ by multiples of p requires estimation of δ at non-integer cycles, and some form of interpolation. Data fits using cubic splines minimize curvature and exhibit non-oscillatory behavior for the data sets under consideration. Other methods that can be used to fit data include penalized splines (P-splines), restricted cubic splines (RCS), natural splines (NS), fractional polynomials (FP), and the like.

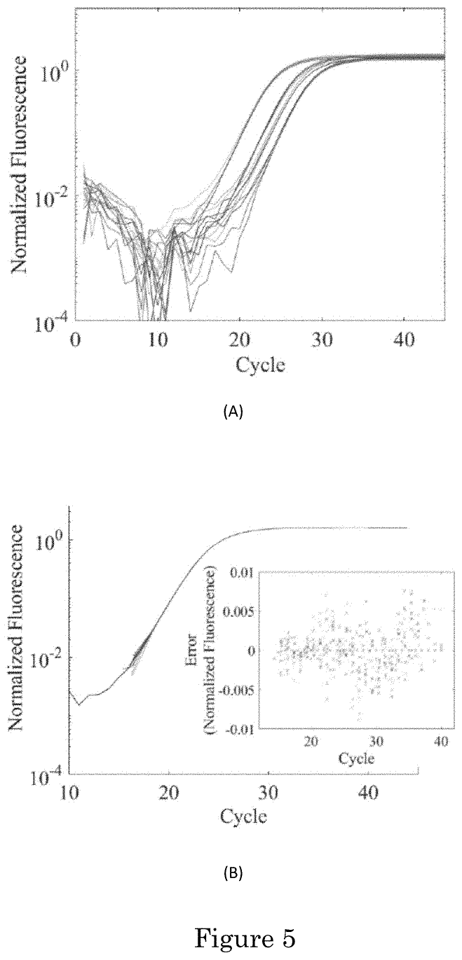

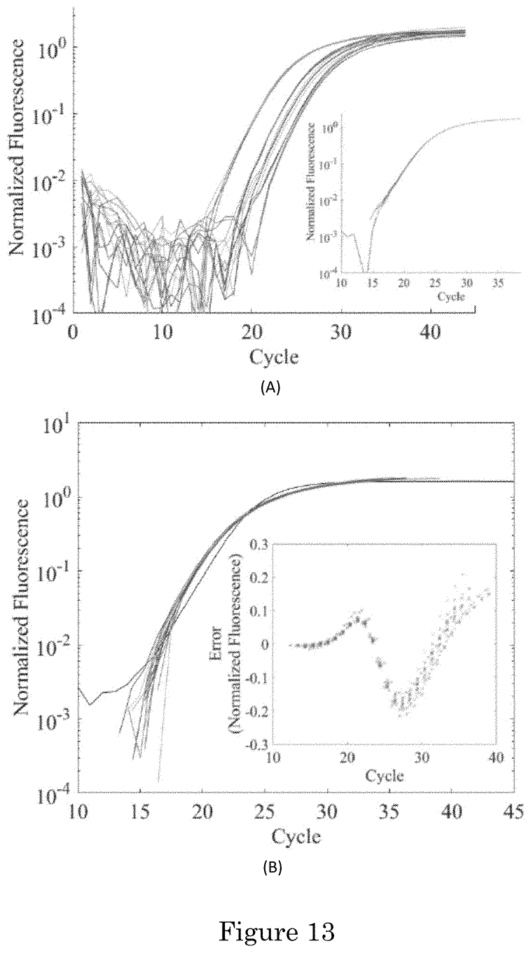

At step 404, validation method 400 applies a constrained optimization approach to determine whether an amplification curve exhibits characteristics that are representative of a true signal by projecting sample signals data obtained at step 106 onto a reference amplification curve obtained at step 402. In one embodiment of the present invention, validation method 400 determines whether each of sample signals obtained at step 106 can be collapsed to master signal obtained at step 402. In some embodiments of the present invention, validation method 400 applies a constrained optimization approach at step 404 to determine whether an amplification curve exhibits characteristics that are representative of a true signal by projecting sample signals data obtained at step 106 onto a library of reference amplification curves to identify a reference amplification curve having a closest match to the given sample. Fluorescence emission signal intensities obtained at step 106 from samples having a particular nucleic acid sequence will only collapse onto a reference amplification signal for the same nucleic acid sequence, i.e. there is amplicon specificity. For example, amplification curves obtained for N1 sequence of covid-19 will collapse only on to reference amplification curve obtained using N1 sequence of covid-19 and N2 sequence of covid-19 will collapse only on to reference amplification curve obtained using N2 sequence of covid-19, as shown in FIG. 13. FIG. 5 illustrates an exemplary analysis applied to RT-qPCR measurements of the N1 fragment of a SARS-CoV-2 RNA construct. FIG. 5(A) shows qPCR curves after background subtraction and FIG. 5(B) shows amplification curves after data collapse with the inset showing error on an absolute scale relative to the master curve.

In one embodiment of the present invention, data collapse is validated using an objective function of the form

[symbol]

(a,c,k,β)=Σ_{N}_{ₘᵢₙ}^{N}^{ₘₐₓ}[δ(n−k)−adₙ−c−βbₙ]² (3)

wherein a, c, β, and k are unspecified parameters, and Nₘᵢₙ and Nₘₐₓ are indices characterizing the cycles for which dₙ is above the noise floor. Minimizing L with respect to its arguments yields the transformation that best matches dₙ onto the reference curve δ(n), wherein δ(n) is an interpolation. In one embodiment of the present invention, δ(n) is a cubic spline. The background signal bn is included in this optimization to ensure that any over or undercorrection of the baseline relative to d is undone.

The quantity Nₘᵢₙ is taken to be the last cycle for which dₙ<μ+3σ, where

$$\begin{matrix}µ = \frac{1}{10} {\sum }_{n = 5}^{14} {d}_{n} & {σ}^{2} = \frac{1}{9} {\sum }_{n = 5}^{14} {( µ - {d}_{n} )}^{2}\end{matrix} \qquad ( 4 )$$

are estimates of the mean and variance associated with the noise η. If Nₘᵢₙ was less than or equal to 30, then Nₘₐₓ is set to 37; else, Nₘₐₓ is set to 40. While it is generally possible to set Nₘₐₓ to 40 for all data sets, it has been observed in certain scenarios that an amplification curve with a higher nominal C_{q} may saturate faster than δ In such scenarios, it may be necessary to decrease Nₘₐₓ so that the interval [Nₘᵢₙ, Nₘₐₓ] is within the domain of cycles spanned by δ Except in scenarios noted above, a meaningfully change may not be observed if such restrictions are imposed for all curves with nominal C_{q} values less than or equal to 30.

The objective function of Eq. (7) is minimized subject to the following constraints that ensure the solution provides fidelity of the data collapse.

−3σ−μ≤c≤3σ+μ (5a)

−3σ−μ·β≤3σ+μ (5b)

−3σ−μ≤c+β≤3σ+μ (5c)

aₘᵢₙ≤a≤aₘₐₓ (5d)

−10≤k≤40 (5e)

τ≤ad_{N}_{ₘₐₓ}+c+βb_{N}_{ₘₐₓ} (5f)

|δ(n−k)−adₙ−c−βbₙ|≤ [symbol] (5g)

Inequalities shown in Eqs. (5a)-(5c) require that the constant offset, noise correction, and linear combination thereof be within the 99% confidence interval of the noise-floor plus any potential offset in the mean (which should be close to zero). Inequality of Eq. (5d) prohibits the multiplicative scale factor from adopting extreme values that would make noise appear to be true exponential growth. In one embodiment, aₘᵢₙ=0.7 and aₘₐₓ=1.3 when inequality of Eq. (5d) prohibits the multiplicative scale factor from adopting extreme values that would make noise appear to be true exponential growth. In some embodiments, a range of admissible values of a corresponds to the maximum variability in the absolute number of reagents per well, which is partially controlled by pipetting errors. Inequality shown in Eq. (5e) controls the range of physically reasonable horizontal offsets. Eq. (5f) requires that the last data-point of ad_{Nmax} be above some threshold z, and Eq. (5g) requires that the absolute error between the reference and scaled curves be less than or equal to [symbol] . In an idealized measurement, [symbol] may be set to be equal to 3σ, but in multichannel systems, imperfections in demultiplexing and/or inherent photodetector noise can introduce additional uncertainties that limit resolution. In another exemplary embodiment of the present invention, inequality show in Eq. (5g) may be expressed in a differentiable form as two separate inequalities.

The optimal transformation parameters a_{*}, c_{*}, k_{*}, and β_{*} determined by minimizing the objective function represented by Eq. (2) is used to define the transformed signal as represented by Eq. (6).

d_{*}(x)=a_{*}dₓ₊ₖ_{_{*}}+c_{*}+β_{*}bₓ₊ₖ_{_{*}} (6)

where x+k_{*} is required to be an integer in the interval [Nₘᵢₙ, Nₘₐₓ].

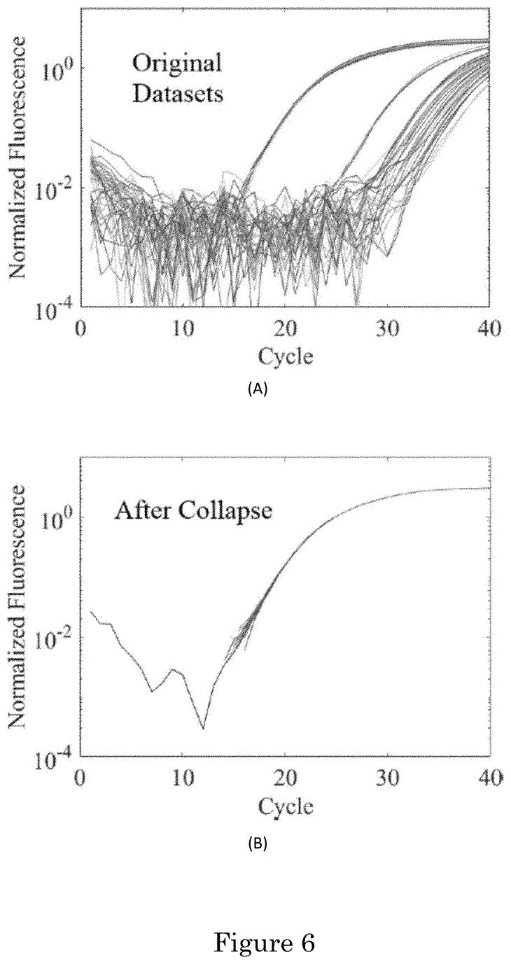

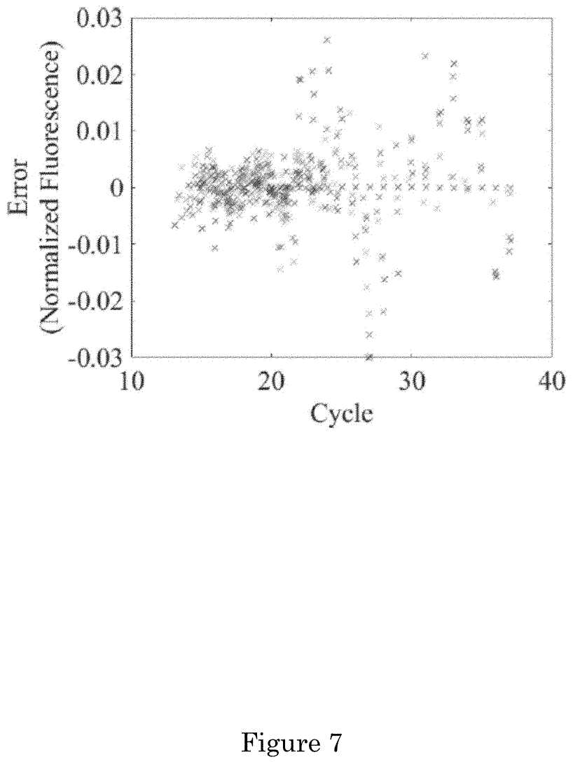

FIG. 6 illustrates exemplary data collapse using optimization methods in accordance with an embodiment of the present invention. FIG. 6(A) illustrates a collection of 43 curves having C_{q} values of less than 37 according to a threshold of 0.1. FIG. 6(B) illustrates the data sets shown in FIG. 6(A) after collapse onto the first curve from the left set as the reference amplification curve. In FIG. 6, during optimization of data collapse, [symbol] is set to be equal to 0:03, which corresponds to roughly 1% of the full scale of the measurement. FIG. 7 illustrates differences between the reference and transformed curves for the collection of datasets in FIG. 6. FIG. 7 illustrates that the errors are less than 0.03 on the normalized fluorescence scale down to the noise floor. This corresponds to less than 1% disagreement relative to the maximum scale. FIG. 6 and FIG. 7 further demonstrates the validity of Eq. (6) for a collection of datasets using a threshold τ=0:05 and having an agreement in fluorescence values of about 0.01, which is more than a decade below typical threshold values used to compute C_{q} for this amplification chemistry. In some embodiments of the present invention, the validated data obtained by data projected or collapsed on to the reference curve is plotted to obtain a validated reference amplification curve. The validated referenced can then be added to the library of reference amplification curves.

At step 406, measurement sensitivity for fluorescence emission signal intensities obtained at step 106 is improved by decreasing fluorescence detection threshold. Embodiments of method 100 in accordance with the present invention recognizes that an advantage of the constrained optimization approach used in 404, as specified in Eqs. (3)-(5g), is the ability to determine when the data set gives rise to a consistent set of constraints. More particularly, inequality shown in Eq. (5g) requires that the transformed signal be within a noise-threshold of the reference for an observable exponential growth.

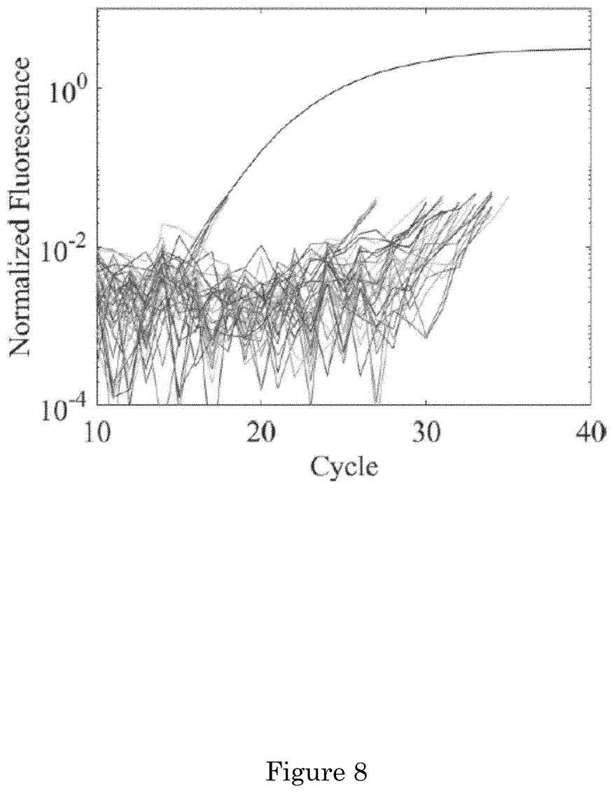

In an exemplary embodiment of the present invention, data set shown in FIG. 8 is used to demonstrate that a non-empty feasible region of the constraints provides a necessary and sufficient condition for determining which data sets have behavior that can be considered statistically meaningful, which in turn can be used to lower the fluorescence thresholds.

In data set of FIG. 8, all data points above a normalized fluorescence value of 0.05 are removed. In this exemplary embodiment, a normalize fluorescence value of 0.05 represents a factor of four below the automated value used by the instrument. The affine transformation according to Eqs. (3)-(5g) is repeated for the last six data points and by setting [symbol] =3 and τ=,μ+6σ in Eq. (5f). In this exemplary embodiment, the value of [symbol] is determined entirely by the noise-floor because a significant spectral overlap is not anticipated at such low fluorescence values.

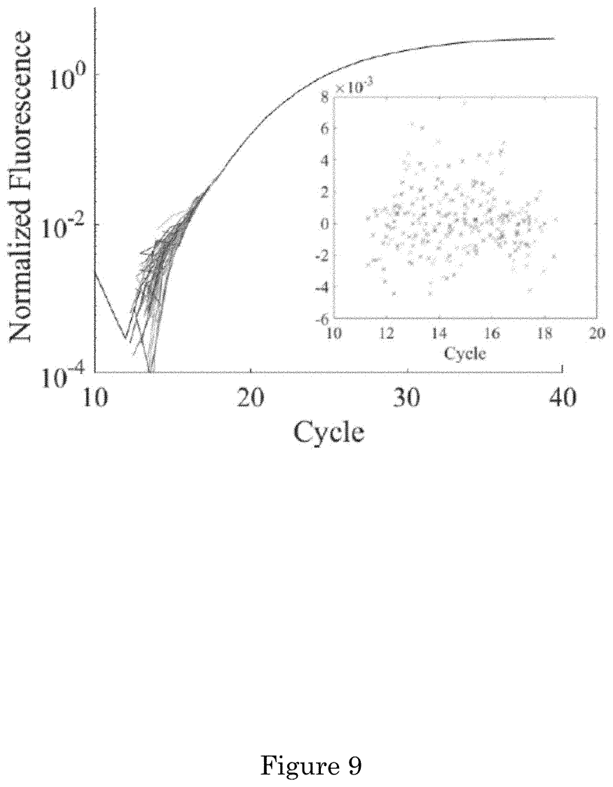

FIG. 8 illustrates a plot including truncated data used to test for feasibility of data collapse using a lower threshold. In FIG. 8, the solid curve is the master curve and the remaining curves have been truncated at the last cycle for which they are below a threshold of 0.05. FIG. 9 illustrates data collapse of the amplification curves shown in FIG. 8. In FIG. 9, the variation in the data below cycle 15 is an artifact of the logarithmic scale that reflects the magnitude of the background noise, and the inset shows that the errors relative to the master curve are less than 10⁻². Further, in FIG. 9, for fluorescence values between 10⁻² and 0.05, the transformed curves are nearly indistinguishable. As shown in FIG. 9, data collapse is achieved using the tightened uncertainty threshold given in terms of the noise-floor and errors relative to the reference curve are less than 0.01 on the normalized fluorescence scale.

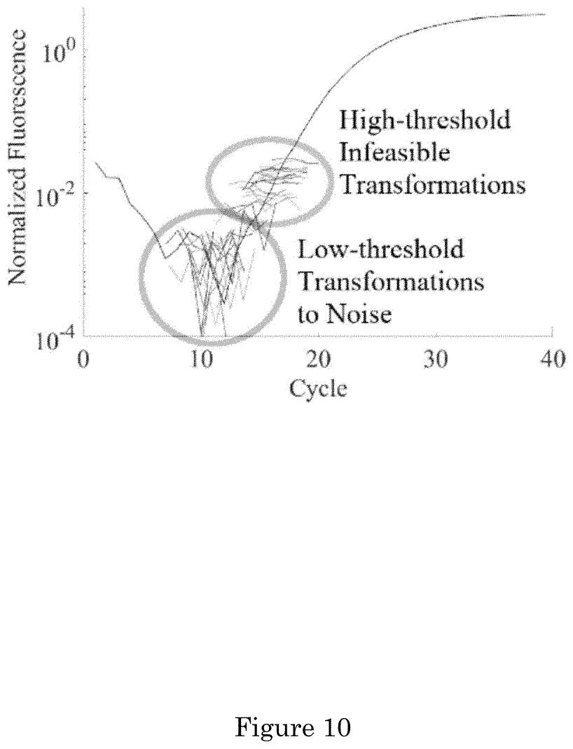

FIG. 10 illustrates exemplary transformations of non-template control data for τ=0 (low-threshold) and τ=μ+5σ (high-threshold), where σ was computed individually for each non-template control. FIG. 10 demonstrates that optimization of the transformation parameters does not generate false positives when the non-template control datasets are baseline-corrected using the methods used on the amplification curves in accordance with an embodiment of the present invention. When τ is small, optimization of transformation parameters maps the non-template controls into the background of the reference curve, thereby illustrating the role of inequality shown in (5f). When τ is large, the optimization of transformation parameters are infeasible, which suggests that there is no transformation satisfying the constraints.

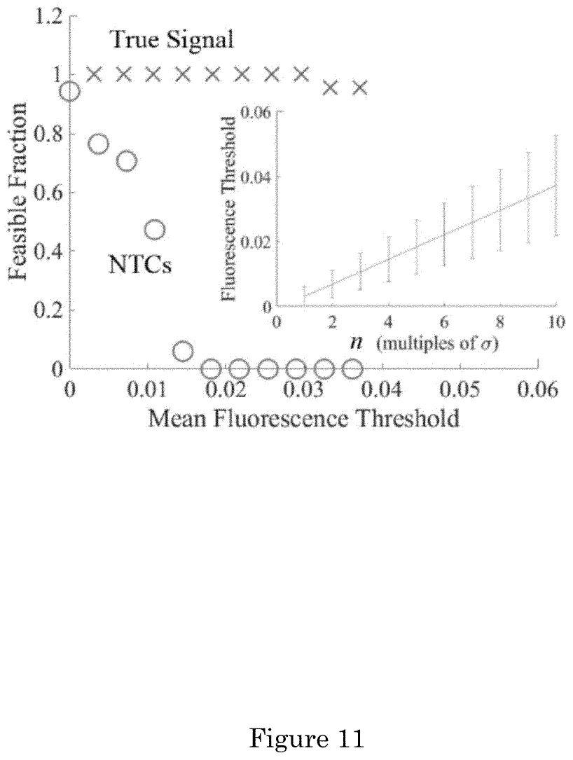

FIG. 11 illustrates feasibility of transforming amplification curves and non-template controls as a function of the mean threshold r. In FIG. 11, the mean value was estimated by setting τ=μ+nσ for n=1, 2, . . . , 10 for each amplification curve and averaging over the corresponding realizations of τ for a fixed n. This process was repeated separately for the non-template controls, including n=0. Further, FIG. 11 shows the average of τ values with one-standard-deviation confidence intervals for each value of n for the amplification curves. In FIG. 11, a setting of 5σ≤τ−μ<8σ yields neither false negatives nor false positives.

Referring back to FIG. 1, output from the data collapse in signal validation step at 110 is used to quantify the initial amount of nucleic acid in the sample at step 112. Quantification method at step 112 leverages a universal property of qPCR; under general conditions, all amplification curves are the same up to an affine transformation. In an embodiment of the present invention, an affine transformation includes a multiplicative, vertical scaling factor and a horizontal shift. In one embodiment of the present invention, affine transformation includes applying affine parameters for a linear transformation and a translation to the data obtained from validation step at 110. Exemplary linear transformations include vertical scaling. Exemplary translations include horizontal shift and vertical shift. In another embodiment of the present invention, affine transformation includes applying affine parameters represented by a polynomial function of a variable to apply a multiplicative factor and a horizontal shift to the data obtained from validation step at 110.

A framework for quantification method at step 112 is based on a generic formulation of a PCR measurement. In this framework, dₙ represents the number of DNA strands at the nth amplification cycle, which, in a noiseless environment, is taken to be proportional to the fluorescence signal measured by the instrument. The outcome of a completed PCR measurement is represented by a vector of the form d=(d₁, d₂, . . . , d_{N}), where N is the maximum cycle number. It is also assumed that d=d(x, y) is a function of the initial template copy number x and the numbers of all other reagents denoted by y.

In one embodiment of the present invention, a framework for quantification method at step 112 requires the following assumptions. First, the framework requires that y be a scalar. This assumption implies that there is a single experimental variable (in addition to initial DNA copies) that controls the progression of the reactions. This condition may be satisfied if all samples to be analyzed include either a single limiting reagent (e.g. primers) or multiple limiting reagents in the same relative concentrations.

Second, the framework requires that there be a p>1 such that a p-fold increase in the initial template number shifts the PCR curve to the left by one cycle. Within a framework in accordance with embodiments of the present invention, such p-fold increase in initial template shift may be represented by

d_{n-q}(p^{q},y)=dₙ(1,y) (7)

In embodiments of the present invention, an increase in template shift as represented by Eq. (7) only requires the amplification efficiency to remain constant over some initial set of cycles qₘₐₓ corresponding to the maximum initial template copy number expected in any given experimental system. In other embodiments of the present invention, an increase in template shift is represented by the following.

d_{n-log}_{ₚ}_{(q/q′)}(q,N)=dₙ(q′,N) (8)

A framework for quantification method at step 112 also requires signal generation be a linear process. This requirement suggests that (i) each sample (e.g. in a well-plate) may be considered to include multiple sub-samples such that the relative fractions of initial DNA and reagents is in proportion to their volumes, and (ii) the total signal generated by a sample is equal to the sum of signals generated by these sub-samples as if they had been separated into different wells. The linearity assumption due to partitioning of an initial template copy and reagent numbers into sub-samples may be represented by the following for any k>0.

dₙ(k,kN)=kdₙ(1,N) (9)

In alternate embodiments of the present invention, a framework for quantification method at step 112 including two values, (x, y) and (χ, γ), for initial template copy number and the numbers of all other reagents, may be represented by the following.

$$\begin{matrix}{d}_{n} ( χ , γ ) = ( γ / y ) {d}_{n} ( χ y / γ , y ) \\ = ( γ / y ) {d}_{n - {log}_{p} [ χ y / ( γ x ) ]} ( x , y ) \\ = a {d}_{n - b} ( x , y )\end{matrix} \qquad ( 10 )$$

Eq. (10) suggests that all PCR signals are the same up to a multiplicative factor a=γ/y and horizontal shift b=logₚ[χy/(χx)] when above assumptions are applied, as required by a framework for quantification method at step 112 in accordance with embodiments of the present invention. This universal property applies irrespective of the actual shape of the amplification curve and under a few generic assumptions. Further, an amplification efficiency does not play a role in the above analysis, as suggested in Eq. (10).

Methods in accordance with embodiments of the present invention has several advantages over previous methods for background subtraction and data analysis. More particularly, method 100 for determining quantity of target nucleic acid in a biological sample in accordance with embodiments of the present invention has more flexibility. More specifically, the generality of the assumptions underpinning Eq. (10) suggests that a master curve may be useful for characterizing qPCR data irrespective of when or where the data was collected. Such universality is advantageous because it facilitates transfer of analyses between laboratories without the need to generate independent master curves. Independent master curves could be developed once with the creation of an assay and used as a type of standard reference data. Such approaches could further harmonize analyses across laboratories and thereby reduce uncertainty in qPCR testing. It has also been discovered that a single master curve can be used for accurate data collapse over measurements from a timeframe spanning a number of years and that the use of a master curve is backwards and forward compatible. Moreover, a library of different master curves can be used to identify a closest match to a given sample to determine the genetic sequence without the need for direct sequencing experiments. Failure to collapse to a member of the library can indicate a new genetic strain (e.g. of a virus) or indicate quality control problems with the amplification.

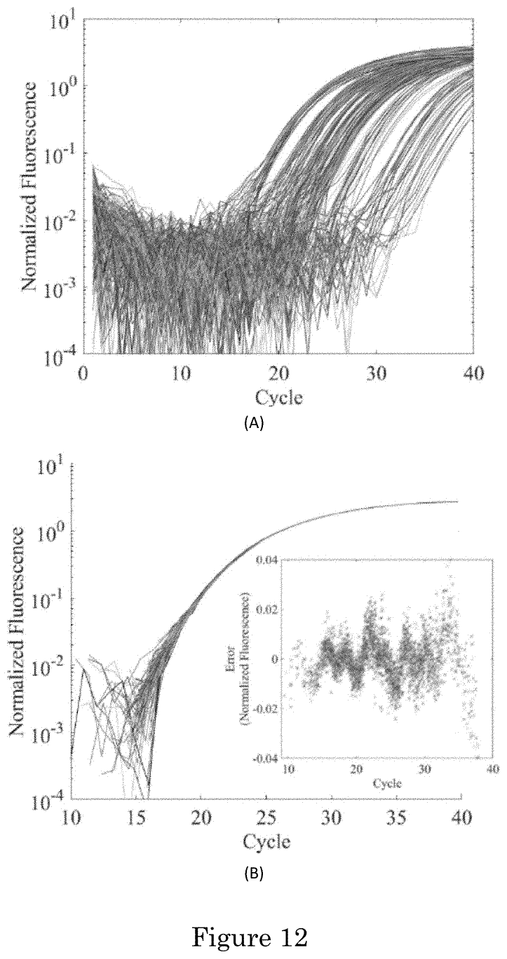

In an exemplary embodiment of the present invention using method 100 for determining quantity of target nucleic acid in a biological sample, analyses were performed on 223 datasets collected over 3.5 years from samples having the same nucleic acid sequence and amplification chemistry. A single low C_{q} curve measured was chosen at random from this set as a master curve, and data collapse was performed on the remaining 222 using optimization methods represented by Eqs. (3)-(5g). FIG. 12 illustrates the results of affine analysis performed on the 223 exemplary datasets spanning 3.5 years. FIG. 12(A) shows 223 amplification curves after background subtraction and FIG. 12(B) shows all datasets collapsed onto one of the amplification curves.

The absolute errors after transformation are also shown in FIG. 12(B), which suggests that the data collapse is accurate to within about 1% of the full scale for nearly all of the measurements. In the affine analysis shown in FIG. 12, [symbol] is set to 0.04 and the minimum and maximum values of a was set to be 0.1 and 3 to account for large variations in peak fluorescence.

Reference now to the specific examples which follow will provide a clearer understanding of systems in accordance with embodiments of the present invention. The examples should not be construed as a limitation upon the scope of the present invention.

Example Application to SARS-CoV-2 RNA

A. SARS-CoV-2 RNA Constructs

A method in accordance with an embodiment of the present invention was applied to RT-qPCR measurements of the N1 and N2 fragments of SARS-CoV-2 RNA. The underlying samples were derived from an in-house, in-vitro transcribed RNA fragment containing approximately 4000 bases of SARS-CoV-2 RNA sequence. This non-infectious fragment contains the complete N gene and E gene, as well as the intervening sequence.

Neat samples of this material were diluted 1:100, 1:500, 1:1000 and 1:1500 in RNA Storage Solution (Thermo Fisher) with 5 ng/μL Jurkat RNA (Thermo Fisher) prior to being run for qPCR. qPCR measurements were performed using the 2019-nCoV CDC Assays (IDT). The N1 and N2 targets on the N gene were measured. Each reaction consisted of 8.5 μL water, 5 TaqPath RT-qPCR Master Mix, 1.5 μL of the IDT primer and probe mix for either N1 or N2, and 5 μL of sample setup in a 96-well optical qCPR plate (Phoenix) and sealed with optical adhesive film (VWR). After sealing the plate, it was briefly centrifuged to eliminate bubbles in the wells. qPCR was performed on an Applied Biosystems 7500 HID instrument with the following thermal cycling protocol: 25 degree C. for 2 minutes, 50 degree C. for 15 minutes, 95 degree C. for 2 minutes followed by 45 cycle of 95 degree C. for 3 seconds and 55 degree C. for 30 seconds. Data was collected at the 55 degree C. stage for 30 seconds for each of the cycles across all wells. Upon completion of every run, data was exported into a spreadsheet for further analysis using Matlab™.

B. Analysis of RT-qPCR Measurements

Data analysis proceeded using non-template controls in lieu of extraction blanks for the background signal bn. FIG. 5 shows the results of this analysis applied to the N1 fragment of a SARS-CoV-2 RNA construct. The level of agreement between curves after data collapse confirms that these signals are virtually identical up to an affine transformation. FIG. 13 shows analogous results for N2 fragment of a SARS-CoV-2 RNA construct. FIG. 13 also illustrates that it not feasible to transform the N2 amplification curves onto the N1 master curve in the bottom plot; the N1 master curve is different in shape from its N2 counterparts. This demonstrates that while the master curve may be transferable across laboratories, it is still specific to the amplification chemistry and target under consideration.

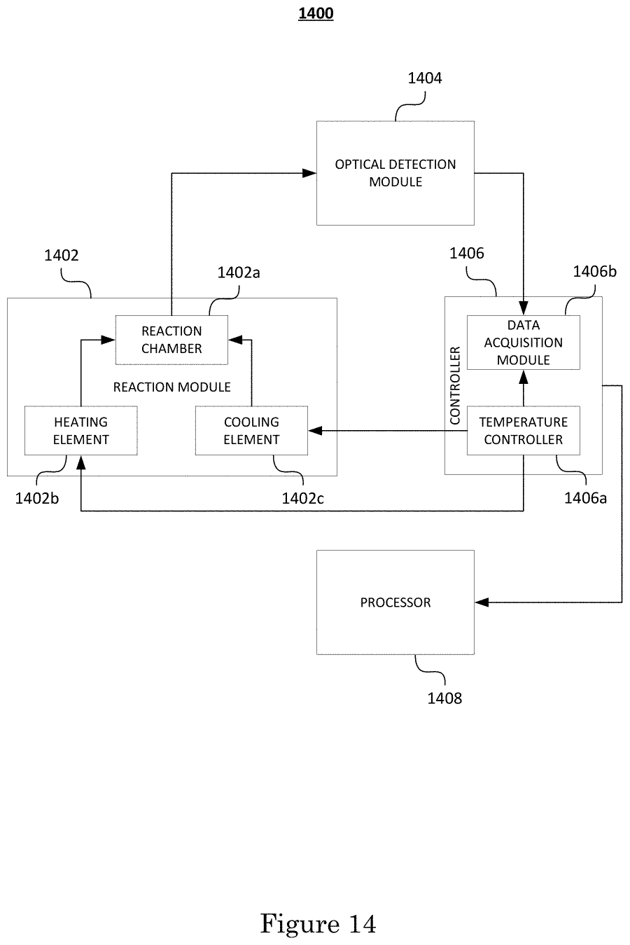

FIG. 14 illustrates an exemplary system 1400 for performing quantitative PCR in conjunction with the quantitation method in accordance with embodiments of the present invention. In one aspect, the system 1400 comprises a reaction module 1402, an optical detection module 1404, a controller 1406 and a processor 1408, which may be interconnected or networked by way of a communications medium to substantially automate the analysis.

Reaction module 1402 receives the samples and thermally cycles the samples to precise temperatures to promote nucleotide denaturation, annealing, and then polymerase-mediated extension for each round of nucleic acid amplification. In one embodiment, reaction module includes a reaction chamber 1402a coupled to a heating element 1402b and a cooling element 1402c configured to thermally cycle the sample in through controlled heating and cooling steps executed over designated time intervals. In one embodiment of the present invention, reaction chamber 1402a can include a block with plurality of reactions wells having a predetermined reaction volume. In one example, a reaction chamber may be a 96-well block with reaction volumes ranging from 1 to 125 μl, a 384-well block with reaction volumes in the pico to nanoliter range. In some embodiments, temperature controller 1406b uses a solid-state active heat pump that transfers heat from one side of reaction chamber to the other against a temperature gradient with the consumption of electrical energy. In other embodiments of the present invention, temperature control is achieved by suspending tubes in reaction chambers and circulating air having a predetermined temperature for time periods as required for PCR.

Optical detection module 1404 determines the presence of target nucleic acid sequence in the sample by detecting and measuring fluorescence generated in the presence of a fluorescent reporter, such as a DNA-binding dye or labeled probe, for each amplification reaction and transmits the fluorescence data to controller 1406. Optical detection module 1404 can include a wide variety of optics systems that use a combination of light sources, filters, and detectors to measure the amount of fluorescence that is present in the amplification reactions. Exemplary light sources that can be used include light-emitting diodes (LEDs), halogen lamp, laser, and the like. Exemplary detectors that can be used include a photodiode, a charge coupled device (CCD), a photomultiplier tube, and the like.

Controller 1406 is coupled to reaction module 1402 and optical detection module 1404, and may be configured to communicate with each module of the system and coordinate system-wide activities to facilitate the automated quantitative PCR analysis. In one embodiment of the present invention, controller 1406 is configured to communicate with each module of the system for protocol setup, plate setup and data collection. Protocol setup may include specifying the denaturation, annealing, and extension parameters, the number of repeated cycles, and the steps at which data are to be collected. Plate setup may include identifying the contents of each well so that data can be properly analyzed once collected and designate the wells to be read.

Controller 1406 incudes a temperature controller 1406a that is coupled to heating element 1402b and cooling element 1402c of reaction module 1402 and configured to regulate the temperature of the samples in reaction chamber 1402a. Controller 1406 incudes a data acquisition module 1406b to record the fluorescence data for each reaction over the specified time course. Data acquisition module 1406b may store the data in numerous different forms and configurations including tables, charts, arrays, spreadsheets, databases, and the like.

Processor 1408 is coupled to controller 1406 to receive fluorescence data from data acquisition module 1406b, plots the fluorescence intensity of the reaction mixture in each well versus the reaction cycle, and performs quantitation method 100 in accordance with embodiments of the present invention. Once a run is complete, processor 1408 performs a background subtraction, signal validation and quantifies the initial amount of nucleic acid sequence in the sample using quantitation method 100 in accordance with various embodiments of the present invention. Quantitation method 100 may be implemented using one or more computer program or modules which comprise functions designed to manipulate the data and generate requested information. In one aspect, the processor 1408 is designed to operate in a user-independent manner where all of the calculations and analytical tasks are performed without the need for the user to manually assess or interpret the data.

Systems and methods for determining quantity of target nucleic acid in a biological sample in accordance with one or more embodiments of the present invention can be adapted to a variety of configurations. It is thought that systems and methods for determining quantity of target nucleic acid in a biological sample in accordance with various embodiments of the present invention and many of its attendant advantages will be understood from the foregoing description and it will be apparent that various changes may be made without departing from the spirit and scope of the invention or sacrificing all of its material advantages, the form hereinbefore described being merely a preferred or exemplary embodiment thereof.

Those familiar with the art will understand that embodiments of the invention may be employed, for various specific purposes, without departing from the essential substance thereof. The description of any one embodiment given above is intended to illustrate an example rather than to limit the invention. This above description is not intended to indicate that any one embodiment is necessarily preferred over any other one for all purposes, or to limit the scope of the invention by describing any such embodiment, which invention scope is intended to be determined by the claims, properly construed, including all subject matter encompassed by the doctrine of equivalents as properly applied to the claims.

What is claimed is:

1. A method for determining a quantity of a target nucleic acid in a sample, the method comprising:
performing a first amplification reaction on a plurality of control samples;
receiving a plurality of first optical signals as a function of a first cycle number for the plurality of the control samples, wherein the plurality of the first optical signals comprises a plurality of first background signals;
performing a second amplification reaction on the sample;
receiving a plurality of second optical signals as a function of a second cycle number for the target nucleic acid in the sample, wherein the plurality of the second optical signals comprise at least one of the plurality of the first background signals and a plurality of second background signals;
optimizing, using a processor, the plurality of the first background signals from the plurality of the first optical signals for each of the plurality of the control samples, wherein the optimizing the plurality of the first background signals comprises subtracting the plurality of the first background signals from the plurality of the second optical signals received to obtain a mean baseline, wherein the mean baseline corresponds to the second cycle number such that the plurality of the second optical signals corresponds to a predetermined value;
validating, using the processor, each of the plurality of the second optical signals against at least one of a plurality of reference amplification curves; and
computing, using the processor, the quantity of the target nucleic acid in the sample from the validated plurality of the second optical signals obtained as the function of the second cycle number for the target nucleic acid in the sample, wherein computing the quantity of the target nucleic acid in the sample from the validated plurality of the second optical signals comprises performing an affine transformation to apply at least one of a linear transformation and a translation on the validated plurality of the second optical signals.

2. The method of claim 1, wherein the receiving the plurality of the first optical signals as the function of the first cycle number comprises:
detecting the plurality of the first optical signals for each of the plurality of the control samples at each cycle of the first amplification reaction, wherein each cycle of the first amplification reaction corresponds to the first cycle number; and
plotting the plurality of the first optical signals detected as the function of the first cycle number for each of the plurality of the control samples to obtain a first amplification curve, wherein the first amplification curve represents a ratio of each of the plurality of the first optical signals to a passive reporter dye optical signal as the function of the first cycle number.

3. The method of claim 1, wherein the receiving the plurality of the second optical signals as the function of the second cycle number comprises:
detecting the plurality of the second optical signals for the target nucleic acid in the sample at each cycle of the second amplification reaction, wherein each cycle of the second amplification reaction corresponds to the second cycle number; and
plotting the plurality of the second optical signals detected as the function of the second cycle number for the target nucleic acid in the sample to obtain a second amplification curve, wherein the second amplification curve represents a ratio of each of the plurality of the second optical signals to a passive reporter dye optical signal as the function of the second cycle number.

4. The method of claim 1, wherein the validating each of the plurality of the second optical signals against the at least one of the plurality of the reference amplification curves comprises:
generating at least the one of the plurality of the reference amplification curves, wherein the at least one of the plurality of the reference amplification curves comprises a plurality of third optical signals corresponding to a reference sample, wherein the at least one of the plurality of the reference amplification curves comprises the plurality of the third optical signals corresponding to a background region, an exponential growth region, and a plateau region;
projecting each of the plurality of the second optical signals on to the at least one of the plurality of the reference amplification signals, wherein the projecting each of the plurality of the second optical signals on to the at least one of the plurality of the reference amplification curves comprises determining whether each of the plurality of the second optical signals obtained as the function of the second cycle number for the target nucleic acid in the sample collapses on to the at least one of the plurality of the reference amplification curves; and
determining a threshold value for the plurality of the second optical signals projected on to the at least one of the plurality of the reference amplification curves.

5. The method of claim 4, further comprising plotting the plurality of the second optical signals projected on to the at least one of the plurality of the reference amplification curves to obtain a validated reference amplification curve.

6. The method of claim 1, further comprising detecting the plurality of third optical signals for a plurality of amplicons, wherein each of the plurality of the third optical signals correspond to an optical signal from at least one fluorescent dye.

7. The method of claim 1, wherein the linear transformation comprises scaling, and wherein the translation is selected from a group comprising a horizontal shift and a vertical scaling.

8. The method of claim 1, wherein the control sample is an extraction blank.

9. The method of claim 1, wherein the control sample is a non-template control.

10. The method of claim 1, wherein the validating each of the plurality of second optical signals against at least one of a plurality of reference amplification curves comprises:
generating the plurality of the reference amplification curves, wherein each of the plurality of the reference amplification curves comprises a plurality of third optical signals corresponding to a reference sample, wherein each of the plurality of the reference amplification curves comprises the plurality of the third optical signals corresponding to a background region, an exponential growth region, and a plateau region;
projecting the plurality of the second optical signals onto the plurality of the reference amplification curves, wherein projecting the plurality of the second optical signals onto the plurality of the reference amplification curves comprises determining whether each of the plurality of the second optical signal obtained as the function of the second cycle number for the target nucleic acid in the sample collapses on to at least one of the plurality of reference amplification curves; and
determining a threshold value for the plurality of the second optical signals projected on to the at least one of the plurality of the reference amplification curves.

11. The method of claim 1, wherein the sample is a pre-amplified DNA sample.

12. The method of claim 1, wherein the sample is a pre-amplified RNA sample.

13. The method of claim 1, further comprising normalizing the plurality of the first optical signals and the plurality of the second optical signals, wherein normalizing the plurality of the first and the second optical signals comprises determining a ratio of each of the plurality of the first and the second optical signals to a passive reporter dye optical signal.

14. A method for determining a quantity of a target nucleic acid in a sample, the method comprising:
performing a first amplification reaction on a plurality of control samples;
receiving a first amplification curve representing a plurality of first optical signals as a function of a first cycle number for each of the plurality of control samples at each cycle of the first amplification reaction, wherein the plurality of the first optical signals comprises a plurality of background signals;
performing a second amplification reaction on the sample;
receiving a second amplification curve representing a plurality of second optical signals as a function of a second cycle number for the target nucleic acid in the sample at each cycle of the second amplification reaction;
optimizing, using the processor, the plurality of the background signals from the received plurality of first optical signals for each of the plurality of the control samples, wherein the optimizing the plurality of the background signals comprises subtracting the plurality of the background signals from the plurality of the second optical signals received to obtain a mean baseline, wherein the mean baseline corresponds to the second cycle number such that the plurality of the second optical signals corresponds to a predetermined value;
validating, using the processor, each of the plurality of second optical signals detected against a plurality of reference amplification curves, wherein validating each of the plurality of the second optical signals comprises:
receiving the plurality of the reference amplification curves, wherein each of the plurality of the reference amplification curves comprises a plurality of third optical signals corresponding to a reference sample, wherein each of the plurality of the reference amplification curves comprises the plurality of the third optical signals corresponding to a background region, an exponential growth region, and a plateau region;
projecting each of the plurality of the second optical signals on to the plurality of the reference amplification curves; and
determining a threshold for the plurality of the second optical signals projected onto at least one of the plurality of the reference amplification curves; and
performing, using the processor, an affine transformation on the validated plurality of the second optical signals to determine the quantity of the target nucleic acid in the sample, wherein the affine transformation comprises at least one of a linear transformation and a translation.

15. The method of claim 14, wherein the control sample is an extraction blank.

16. The method of claim 14, wherein the control sample is a non-template control.

17. The method of claim 14, wherein the projecting each of the plurality of the second optical signals on to the plurality of the reference amplification curves comprises determining whether each of the plurality of the second optical signal obtained as the function of the second cycle number for the target nucleic acid in the sample collapses on to at the least one of the plurality of the reference amplification curves.

18. The method of claim 14, wherein the sample is a pre-amplified DNA sample.

19. The method of claim 14, wherein the sample is a pre-amplified RNA sample.

20. A system of determining quantity of a target nucleic acid in a sample, said system comprising:
a reaction chamber for performing a first amplification reaction on a plurality of control samples and a second amplification reaction on the sample;
a detector for detecting a plurality of first optical signals as a function of a first cycle number for the plurality of the control samples at each cycle of the first amplification reaction and a plurality of second optical signals as a function of a second cycle number for the target nucleic acid in the sample at each cycle of the second amplification reaction, wherein the plurality of the first optical signals comprises a plurality of background signals; and
a processor comprising a memory for implementing program instructions of a computer-readable medium, wherein said program instructions comprise:
optimizing the plurality of the background signals from the received plurality of the first optical signals for each of the plurality of the control samples, wherein the optimizing the plurality of the background signals comprises subtracting the plurality of the background signals from each of the plurality of the second optical signals received to obtain a mean baseline, wherein the mean baseline corresponds to the second cycle number such that the plurality of the second optical signals correspond to a predetermined value;
validating each of the plurality of the second optical signals detected against a plurality of reference amplification curves, wherein the validating each of the plurality of the second optical signals comprises projecting each of the plurality of the second optical signals on to the plurality of the reference amplification curves to determine a threshold for the plurality of the second optical signals; and
computing an initial quantity of the target nucleic acid in the sample from the validated plurality of the second optical signals obtained as the function of the second cycle number for the target nucleic acid in the sample, wherein computing the initial quantity of the target nucleic acid in the sample from the validated plurality of the second optical signals comprises performing an affine transformation on the validated plurality of the second optical signals, wherein the affine transformation comprises at least one of a linear transformation and a translation.
